(19) United States
(12) Patent Application Publication
(10) Pub. No.: US 2013/0172747 A1
(43) Pub. Date: Jul. 4, 2013

(54) ESTIMATING MOTION OF PARTICLE BASED ON VECTOR DOPPLER IN ULTRASOUND SYSTEM

(71) Applicant: Samsung Medison Co., Ltd., Gangwon-do (KR)

(72) Inventors: Min Woo KIM, Seoul (KR); Hyoung Jin KIM, Seoul (KR)

(73) Assignee: SAMSUNG MEDISON CO., LTD, Gangwon-do (KR)

(21) Appl. No.: 13/659,528

(22) Filed: Oct. 24, 2012

(30) Foreign Application Priority Data
Dec. 28, 2011 (KR) 10-2011-0144489

(51) Int. Cl. A61B 8/14

(52) U.S. Cl. 600/443

(57) ABSTRACT

There are provided embodiments for estimating the motion of at least one particle by using vector Doppler. In one embodiment, by way of non-limiting example, an ultrasound system comprises: a processing unit configured to form vector information of a target object based on ultrasound data corresponding to the target object, form a plurality of Doppler mode images based on the vector information, set at least one particle on the Doppler mode images based on input information of a user, and estimate a motion of the at least one particle based on the vector information.

### CROSS-REFERENCE TO RELATED APPLICATION

The present application claims priority from Korean Patent Application No. 10-2011-0144489 filed on Dec. 28, 2011, the entire subject matter of which is incorporated herein by reference.

### TECHNICAL FIELD

The present disclosure generally relates to ultrasound systems, and more particularly to estimating the motion of at least one particle by using vector Doppler in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two-dimensional or three-dimensional ultrasound images of internal features of target objects (e.g., human organs).

The ultrasound system may provide ultrasound images of various modes including a brightness mode image representing reflection coefficients of ultrasound signals (i.e., ultrasound echo signals) reflected from a target object of a living body with a two-dimensional image, a Doppler mode image representing velocity of a moving target object with spectral Doppler by using a Doppler effect, a color Doppler mode image representing velocity of the moving target object with colors by using the Doppler effect, an elastic image representing mechanical characteristics of tissues before and after applying compression thereto, etc.

The ultrasound system may transmit ultrasound signals to the living body including a moving target object (e.g., blood flow) and receive ultrasound signals (i.e., ultrasound echo signals) from the living body. The ultrasound system may further form the color Doppler mode image representing velocities of the target object with colors based on the ultrasound echo signals. The color Doppler image may be used to diagnose disease of a blood vessel, a heart and the like. However, the color Doppler image cannot represent an accurate motion of the target object since the respective colors in the color Doppler image indicate the velocity of the target object, which moves forward in a transmission direction of the ultrasound signals and backward in the transmission direction of the ultrasound signals.

To resolve this problem, vector Doppler methods capable of obtaining motion (i.e., velocity and direction) of the target object are used. A cross beam-based method of the vector Doppler methods acquires velocity components of the target object from at least two different directions, and combines the velocity components to form vector information including two-dimensional or three-dimensional direction information and velocity information.

### SUMMARY

There are provided embodiments for estimating the motion of at least one particle by using vector Doppler.

In one embodiment, by way of non-limiting example, an ultrasound system comprises: a processing unit configured to form vector information of a target object based on ultrasound data corresponding to the target object, form a plurality of Doppler mode images based on the vector information, set at least one particle on the Doppler mode images based on input information of a user, and estimate a motion of the at least one particle based on the vector information.

In another embodiment, there is provided a method of estimating the motion of at least one particle, comprising: a) forming vector information of a target object based on ultrasound data corresponding to the target object; b) forming a plurality of Doppler mode images based on the vector information; c) setting at least one particle on the Doppler mode images based on input information of a user; and d) estimating a motion of the at least one particle based on the vector information.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a schematic diagram showing an example of a brightness mode image and a region of interest.

FIG. 3 is a block diagram showing an illustrative embodiment of an ultrasound data acquiring unit.

FIGS. 4 to 7 are schematic diagrams showing examples of transmission directions and reception directions.

FIG. 8 is a schematic diagram showing an example of sampling data and pixels of an ultrasound image.

FIGS. 9 to 12 are schematic diagrams showing examples of performing a receiving beam-forming.

FIG. 13 is a schematic diagram showing an example of setting weights.

FIG. 14 is a schematic diagram showing an example of setting a sampling data set.

FIG. 15 is a flow chart showing a process of estimating motion of at least one particle.

FIG. 16 is a schematic diagram showing an example of the transmission directions, the reception directions, the vector information and an over-determined problem.

FIG. 17 is a schematic diagram showing an example of setting the at least one particle.

FIG. 18 is a schematic diagram showing an example of estimating a streamline.

FIG. 19 is a schematic diagram showing an example of moving the particle.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

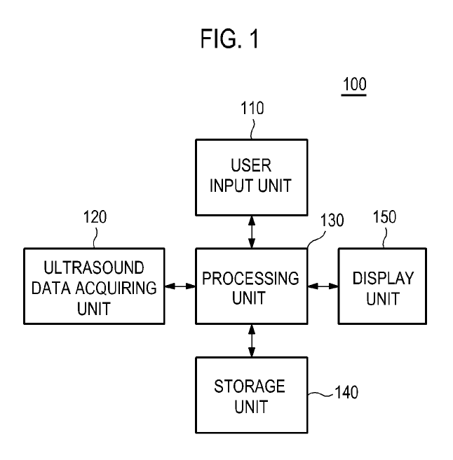

Referring to FIG. 1, an ultrasound system 100 in accordance with an illustrative embodiment is shown. As depicted herein, the ultrasound system 100 may include a user input unit 110.

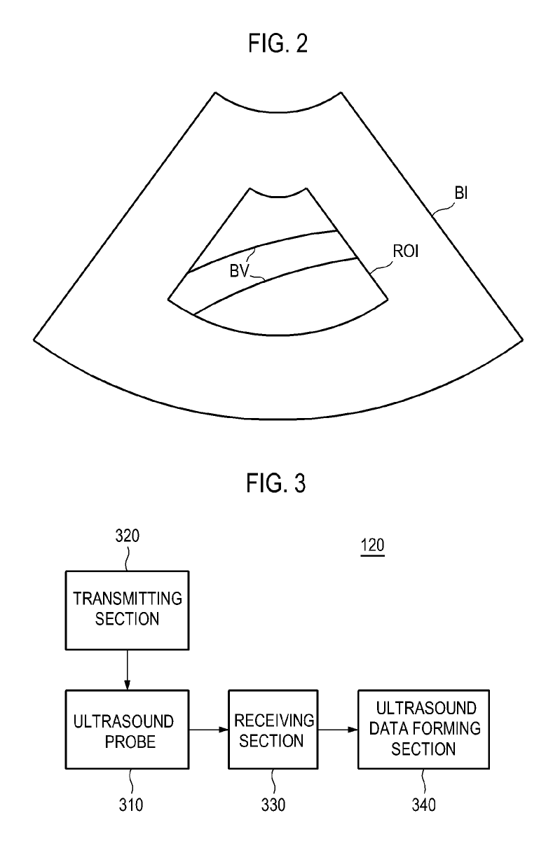

The user input unit 110 may be configured to receive input information from a user. In one embodiment, the input information may include first input information for setting a region of interest ROI on a brightness mode image BI, as shown in FIG. 2. The region of interest ROI may include a color box for obtaining a Doppler mode image. The Doppler mode image may include a vector Doppler image or a color Doppler image. However, it should be noted herein that the Doppler mode image may not be limited thereto. In FIG. 2, a reference numeral BV represents a blood vessel. The input information may further include second input information for setting at least one particle on the Doppler mode image. That is, the second input information may include information for setting the number, position, size, color and shape of the particle, an alignment position between the particles and the like. The user input unit 110 may include a control panel, a track ball, a mouse, a keyboard and the like.

The ultrasound system 100 may further include an ultrasound data acquiring unit 120. The ultrasound data acquiring unit 120 may be configured to transmit ultrasound signals to a living body. The living body may include moving target objects (e.g., blood vessel, heart, blood flow, etc). The ultrasound data acquiring unit 120 may be further configured to receive ultrasound signals (i.e., ultrasound echo signals) from the living body to acquire ultrasound data corresponding to an ultrasound image.

FIG. 3 is a block diagram showing an illustrative embodiment of the ultrasound data acquiring unit. Referring to FIG. 3, the ultrasound data acquiring unit 120 may include an ultrasound probe 310.

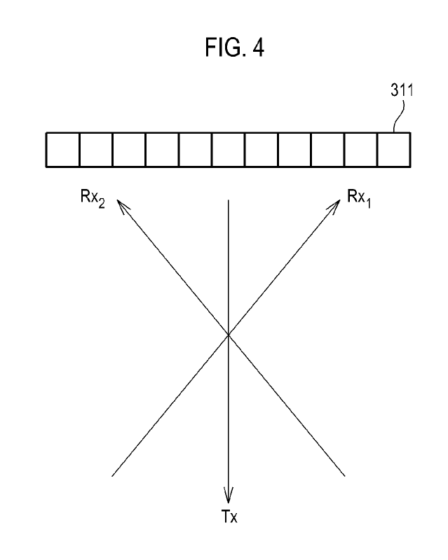

The ultrasound probe 310 may include a plurality of elements 311 (see FIG. 4) for reciprocally converting between ultrasound signals and electrical signals. The ultrasound probe 310 may be configured to transmit the ultrasound signals to the living body. The ultrasound signals transmitted from the ultrasound probe 310 may be plane wave signals that the ultrasound signals are not focused at a focusing point or focused signals that the ultrasound signals are focused at the focusing point. However, it should be noted herein that the ultrasound signals may not be limited thereto. The ultrasound probe 310 may be further configured to receive the ultrasound echo signals from the living body to output electrical signals (hereinafter, referred to as “reception signals”). The reception signals may be analog signals. The ultrasound probe 310 may include a convex probe, a linear probe and the like.

The ultrasound data acquiring unit 120 may further include a transmitting section 320. The transmitting section 320 may be configured to control the transmission of the ultrasound signals. The transmitting section 320 may be further configured to generate electrical signals (hereinafter, referred to as “transmission signals”) in consideration of the elements 311.

In one embodiment, the transmitting section 320 may be configured to generate transmission signals (hereinafter, referred to as “brightness mode transmission signals”) for obtaining the brightness mode image BI in consideration of the elements 311. Thus, the ultrasound probe 310 may be configured to convert the brightness mode transmission signals provided from the transmitting section 320 into the ultrasound signals, transmit the ultrasound signals to the living body, and receive the ultrasound echo signals from the living body to output reception signals (hereinafter, referred to as “brightness mode reception signals”).

The transmitting section 320 may be further configured to generate transmission signals (hereinafter, referred to as “Doppler mode transmission signals”) corresponding to an ensemble number in consideration of the elements 311 and at least one transmission direction of the ultrasound signals (i.e., transmission beam). Thus, the ultrasound probe 310 may be configured to convert the Doppler mode transmission signals provided from the transmitting section 320 into the ultrasound signals, transmit the ultrasound signals to the living body in the at least one transmission direction, and receive the ultrasound echo signals from the living body to output reception signals (hereinafter, referred to as “Doppler mode reception signals”). The ensemble number may represent the number of transmitting and receiving the ultrasound signals to/from a target object.

As one example, the transmitting section 320 may be configured to generate the Doppler mode transmission signals corresponding to the ensemble number in consideration of a transmission direction Tx and the elements 311, as shown in FIG. 4. The transmission direction may be one direction in the range of a direction (0 degree) perpendicular to a longitudinal direction of the elements 311 to a maximum steering direction of the transmission beam.

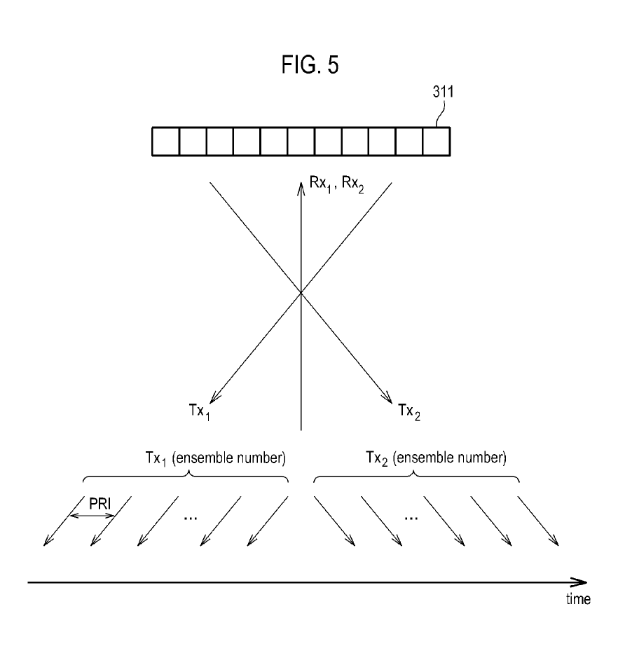

As another example, the transmitting section 320 may be configured to generate first Doppler mode transmission signals corresponding to the ensemble number in consideration of a first transmission direction Tx₁ and the elements 311, as shown in FIG. 5. Thus, the ultrasound probe 310 may be configured to convert the first Doppler mode transmission signals provided from the transmitting section 320 into the ultrasound signals, transmit the ultrasound signals to the living body in the first transmission direction Tx₁, and receive the ultrasound echo signals from the living body to output first Doppler mode reception signals. The transmitting section 320 may be further configured to generate second Doppler mode transmission signals corresponding to the ensemble number in consideration of a second transmission direction Tx₂ and the elements 311, as shown in FIG. 5. Thus, the ultrasound probe 310 may be configured to convert the second Doppler mode transmission signals provided from the transmitting section 320 into the ultrasound signals, transmit the ultrasound signals to the living body in the second transmission direction Tx₂, and receive the ultrasound echo signals from the living body to output second Doppler mode reception signals. In FIG. 5, a reference numeral PRI represents a pulse repeat interval.

In another embodiment, the transmitting section 320 may be configured to generate the brightness mode transmission signals for obtaining the brightness mode image BI in consideration of the elements 311. Thus, the ultrasound probe 310 may be configured to convert the brightness mode transmission signals provided from the transmitting section 320 into the ultrasound signals, transmit the ultrasound signals to the living body, and receive the ultrasound echo signals from the living body to output the brightness mode reception signals.

The transmitting section 320 may be further configured to generate the Doppler mode transmission signals corresponding to the ensemble number in consideration of the at least one transmission direction and the elements 311. Thus, the ultrasound probe 310 may be configured to convert the Doppler mode transmission signals provided from the transmitting section 320 into the ultrasound signals, transmit the ultrasound signals to the living body, and receive the ultrasound echo signals from the living body to output the Doppler mode reception signals. The ultrasound signals may be transmitted in an interleaved transmission scheme. The interleaved transmission scheme will be described below in detail.

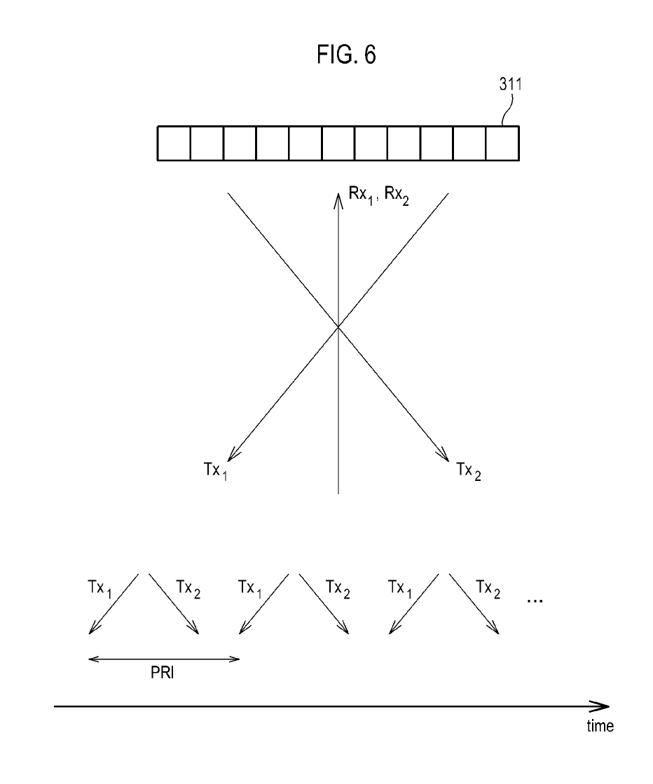

For example, the transmitting section 320 may be configured to generate the first Doppler mode transmission signals in consideration of the first transmission direction Tx₁ and the elements 311, as shown in FIG. 6. Thus, the ultrasound probe 310 may be configured to convert the first Doppler mode transmission signals provided from the transmitting section 320 into the ultrasound signals, and transmit the ultrasound signals to the living body in the first transmission direction Tx₁. Thereafter, the transmitting section 320 may be further configured to generate the second Doppler mode transmission signals in consideration of the second transmission direction Tx₂ and the elements 311, as shown in FIG. 6. Thus, the ultrasound probe 310 may be configured to convert the second Doppler mode transmission signals provided from the transmitting section 320 into the ultrasound signals, and transmit the ultrasound signals to the living body in the second transmission direction Tx₂. The ultrasound probe 310 may be further configured to receive the ultrasound echo signals (i.e., ultrasound echo signals corresponding to first Doppler mode transmission signals) from the living body to output the first Doppler mode reception signals. The ultrasound probe 310 may be further configured to receive the ultrasound echo signals (i.e., ultrasound echo signals corresponding to second Doppler mode transmission signals) from the living body to output the second Doppler mode reception signals.

Thereafter, the transmitting section 320 may be configured to generate the first Doppler mode transmission signals based on the pulse repeat interval, as shown in FIG. 6. Thus, the ultrasound probe 310 may be configured to convert the first Doppler mode transmission signals provided from the transmitting section 320 into the ultrasound signals, and transmit the ultrasound signals to the living body in the first transmission direction Tx₁. The transmitting section 320 may be further configured to generate the second Doppler mode transmission signals based on the pulse repeat interval, as shown in FIG. 6. Thus, the ultrasound probe 310 may be configured to convert the second Doppler mode transmission signals provided from the transmitting section 320 into the ultrasound signals, and transmit the ultrasound signals to the living body in the second transmission direction Tx₂. The ultrasound probe 310 may be further configured to receive the ultrasound echo signals (i.e., ultrasound echo signals corresponding to first Doppler mode transmission signals) from the living body to output the first Doppler mode reception signals. The ultrasound probe 310 may be further configured to receive the ultrasound echo signals (i.e., ultrasound echo signals corresponding to second Doppler mode transmission signals) from the living body to output the second Doppler mode reception signals.

As described above, the transmitting section 320 may be configured to generate the first Doppler mode transmission signals and the second Doppler mode transmission signals corresponding to the ensemble number.

In yet another embodiment, the transmitting section 320 may be configured to generate the brightness mode transmission signals for obtaining the brightness mode image BI in consideration of the elements 311. Thus, the ultrasound probe 310 may be configured to convert the brightness mode transmission signals provided from the transmitting section 320 into the ultrasound signals, transmit the ultrasound signals to the living body, and receive the ultrasound echo signals from the living body to output the brightness mode reception signals.

The transmitting section 320 may be further configured to generate the Doppler mode transmission signals corresponding to the ensemble number in consideration of the at least one transmission direction and the elements 311. Thus, the ultrasound probe 310 may be configured to convert the Doppler mode transmission signals provided from the transmitting section 320 into the ultrasound signals, transmit the ultrasound signals to the living body, and receive the ultrasound echo signals from the living body to output the Doppler mode reception signals. The ultrasound signals may be transmitted according to the pulse repeat interval.

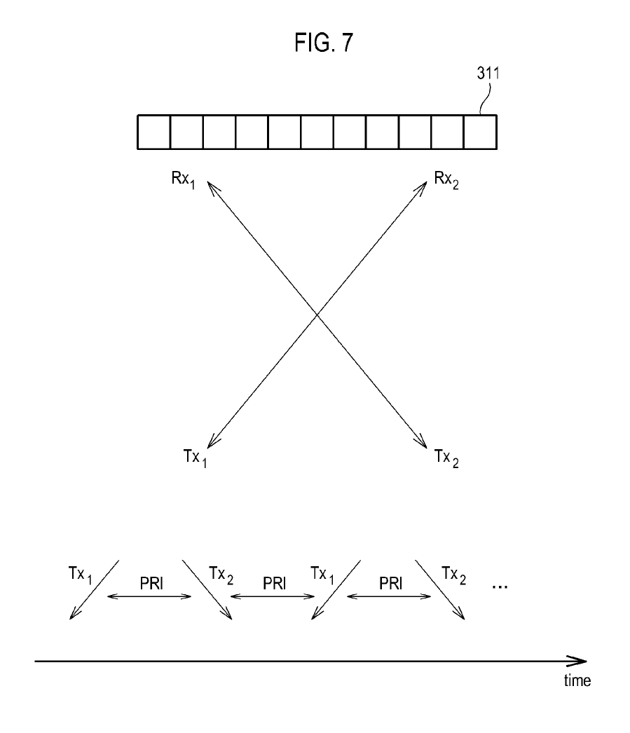

For example, the transmitting section 320 may be configured to generate the first Doppler mode transmission signals in consideration of the first transmission direction Tx₁ and the elements 311 based on the pulse repeat interval, as shown in FIG. 7. As such, the ultrasound probe 310 may be configured to convert the first Doppler mode transmission signals provided from the transmitting section 320 into the ultrasound signals, transmit the ultrasound signals to living body in the first transmission direction Tx₁, and receive the ultrasound echo signals from the living body to output the first Doppler mode reception signals. The transmitting section 320 may be further configured to generate the second Doppler mode transmission signals in consideration of the second transmission direction Tx₂ and the elements 311, based on the pulse repeat interval, as shown in FIG. 7. Thus, the ultrasound probe 310 may be configured to convert the second Doppler mode transmission signals provided from the transmitting section 320 into the ultrasound signals, transmit the ultrasound signals to the living body in the second transmission direction Tx₂, and receive the ultrasound echo signals from the living body to output the second Doppler mode reception signals.

As described above, the transmitting section 320 may be configured to generate the first Doppler mode transmission signals and the second Doppler mode transmission signals corresponding to the ensemble number based on the pulse repeat interval.

Referring back to FIG. 3, the ultrasound data acquiring unit 120 may further include a receiving section 330. The receiving section 330 may be configured to perform an analog-digital conversion upon the reception signals provided from the ultrasound probe 310 to form sampling data of the reception signals. The receiving section 330 may be further configured to perform a reception beam-forming upon the sampling data in consideration of the elements 311 to form reception-focused data. The reception beam-forming will be described below in detail.

In one embodiment, the receiving section 330 may be configured to perform the analog-digital conversion upon the brightness mode reception signals provided from the ultrasound probe 310 to form sampling data (hereinafter, referred to as “brightness mode sampling data”). The receiving section 330 may be further configured to perform the reception beam-forming upon the brightness mode sampling data to form reception-focused data (hereinafter, referred to as “brightness mode reception-focused data”).

The receiving section 330 may be further configured to perform the analog-digital conversion upon the Doppler mode reception signals provided from the ultrasound probe 310 to form sampling data (hereinafter, referred to as “Doppler mode sampling data”). The receiving section 330 may be further configured to perform the reception beam-forming upon the Doppler mode sampling data to form reception-focused data (hereinafter, referred to as “Doppler mode reception-focused data”) corresponding to the at least one reception direction of the ultrasound echo signals (i.e., reception beam).

As one example, the receiving section 330 may be configured to perform the analog-digital conversion upon the Doppler mode reception signals provided from the ultrasound probe 310 to form the Doppler mode sampling data. The receiving section 330 may be further configured to perform the reception beam-forming upon the Doppler mode sampling data to form first Doppler mode reception-focused data corresponding to the first reception direction Rx₁ and second Doppler mode reception-focused data corresponding to the second reception direction Rx₂, as shown in FIG. 4.

As another example, the receiving section 330 may be configured to perform the analog-digital conversion upon the first Doppler mode reception signals provided from the ultrasound probe 310 to form first Doppler mode sampling data corresponding to the first transmission direction Tx₁, as shown in FIG. 5. The receiving section 330 may be further configured to perform the reception beam-forming upon the first Doppler mode sampling data to form the first Doppler mode reception-focused data corresponding to the first reception direction Rx₁. The receiving section 330 may be further configured to perform the analog-digital conversion upon the second Doppler mode reception signals provided from the ultrasound probe 310 to form second Doppler mode sampling data corresponding to the second transmission direction Tx₂, as shown in FIG. 5. The receiving section 330 may be further configured to perform the reception beam-forming upon the second Doppler mode sampling data to form the second Doppler mode reception-focused data corresponding to the second reception direction Rx₂. If the reception direction is perpendicular to the elements 311 of the ultrasound probe 310, then an aperture size of being capable of receiving the ultrasound signals can be a maximum value.

The reception beam-forming may be described with reference to the accompanying drawings.

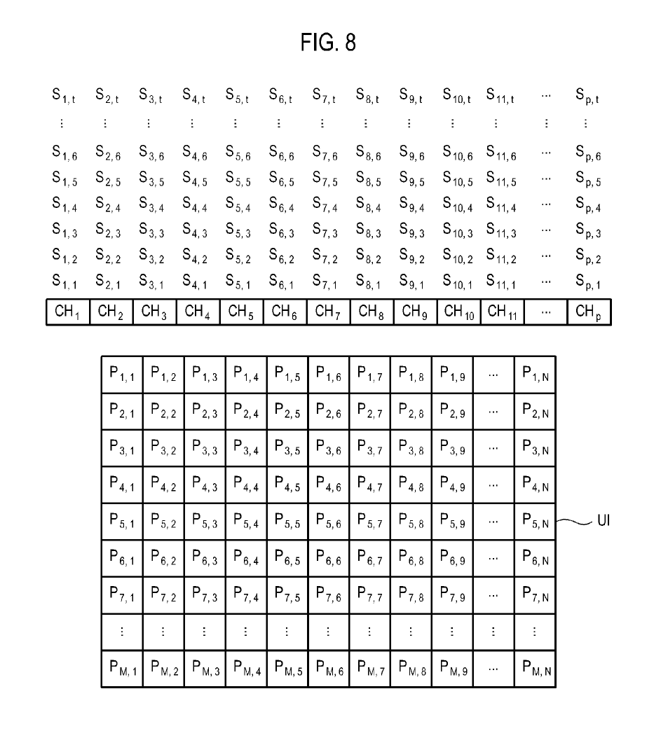

In one embodiment, the receiving section 330 may be configured to perform the analog-digital conversion upon the reception signals provided through a plurality of channels CHₖ, wherein 1≦k≦N, from the ultrasound probe 310 to form sampling data wherein the i and j are a positive integer, as shown in FIG. 8. The sampling data S_{i,j}, may be stored in a storage unit 140. The receiving section 330 may be further configured to detect pixels corresponding to the sampling data based on positions of the elements 311 and orientation of pixels P_{a,b}, wherein of the 1≦a≦M, 1≦b≦N, of the ultrasound image UI with respect to the elements 311. That is, the receiving section 330 may select the pixels that the respective sampling data are used as pixel data thereof, during the reception beam-forming based on the positions of the elements 311 and the orientation of the respective pixels of the ultrasound image UI with respect to the elements 311. The receiving section 330 may be further configured to cumulatively assign the sampling data corresponding to the selected pixels as the pixel data.

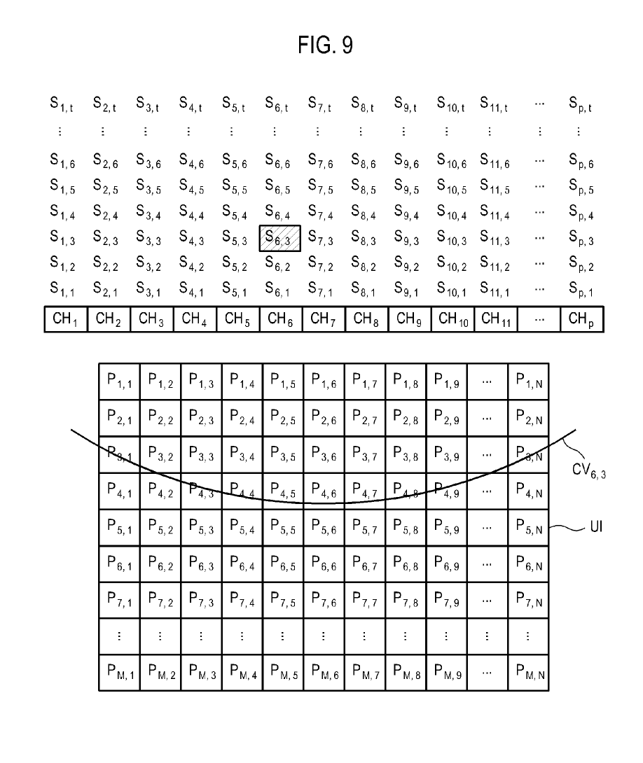

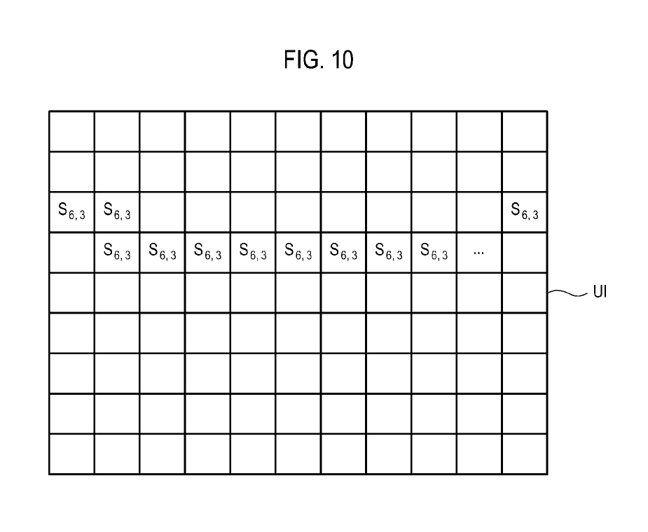

For example, the receiving section 330 may be configured to set a curve (hereinafter, referred to as “reception beam-forming curve”) CV_{6,3} for selecting pixels that the sampling data S_{6,3} are used as the pixel data thereof, during the reception beam-forming based on the positions of the elements 311 and the orientation of the respective pixels of the ultrasound image UI with respect to the elements 311, as shown in FIG. 9. The receiving section 330 may be further configured to detect the pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N} corresponding to the reception beam-forming curve CV_{6,3} from the pixels P_{a,b} of the ultrasound image UI, wherein 1≦a≦M, 1≦b≦N. That is, the receiving section 330 may select the pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N} on which the reception beam-forming curve CV_{6,3} passes among the pixels P_{a,b} of the ultrasound image UI. The receiving section 330 may be further configured to assign the sampling data S_{6,3} to the selected pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N}, as shown in FIG. 10.

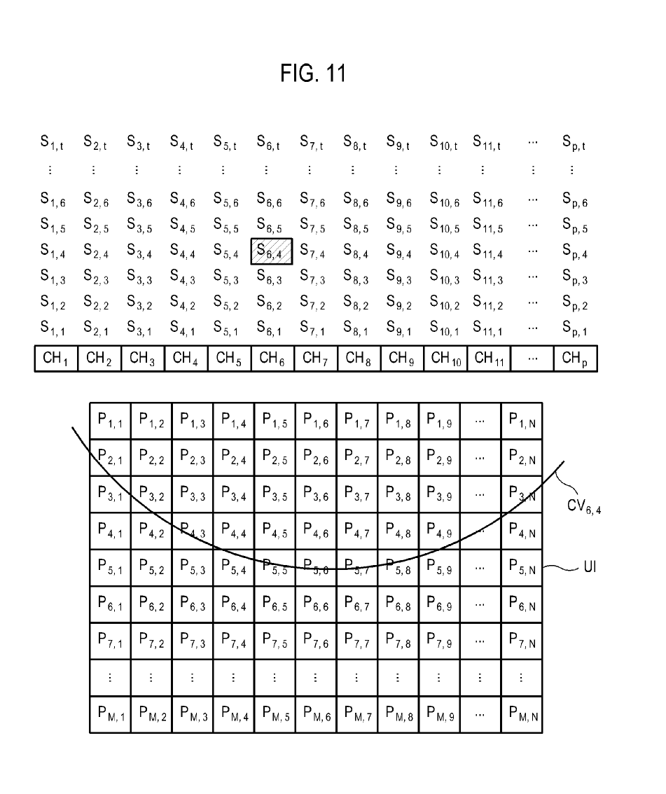

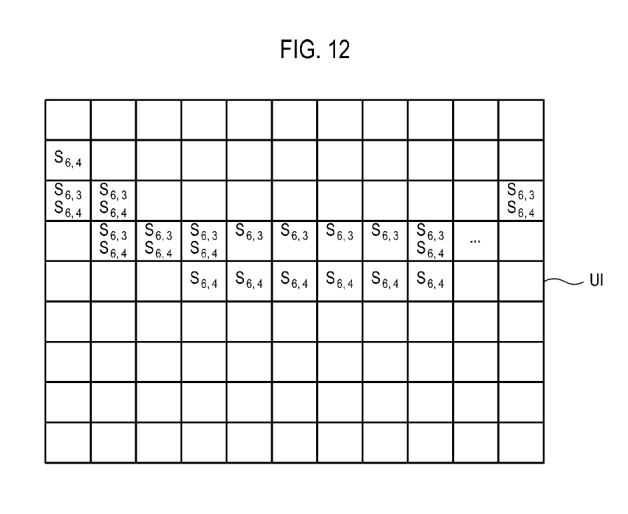

Thereafter, the receiving section 330 may be configured to set a reception beam-forming curve CV_{6,4} for selecting pixels that the sampling data S_{6,4} are used as the pixel data thereof, during the reception beam-forming based on the positions of the elements 311 and the orientation of the respective pixels of the ultrasound image UI with respect to the elements 311, as shown in FIG. 11. The receiving section 330 may be further configured to detect the pixels P_{2,1}, P_{3,1}, P_{3,2}, P_{4,2}, P_{4,3}, P_{4,4}, P_{5,4}, P_{5,5}, P_{5,6}, P_{5,7}, P_{5,8}, P_{4,9}, P_{5,9}, . . . P_{4,N}, P_{3,N} corresponding to the reception beam-forming curve CV₆₄ from the pixels P_{a,b} of the ultrasound image UI. That is, the receiving section 330 may select the pixels P_{2,1}, P_{3,1}, P_{3,2}, P_{4,2}, P_{4,3}, P_{4,4}, P_{5,4}, P_{5,5}, P_{5,6}, P_{5,7}, P_{5,8}, P_{4,9}, P_{5,9}, . . . P_{4,N}, P_{3,N} on which the reception beam-forming curve CV_{6,4} passes among the pixels P_{a,b} of the ultrasound image UI. The receiving section 330 may be further configured to assign the sampling data S_{6,4} to the selected pixels P_{2,1}, P_{3,1}, P_{3,2}, P_{4,2}, P_{4,3}, P_{4,4}, P_{5,4}, P_{5,5}, P_{5,6}, P_{5,7}, P_{5,8}, P_{4,9}, P_{5,9}, . . . P_{4,N}, P_{3,N}, as shown in FIG. 12. In this way, the respective sampling data, which are used as the pixel data, may be cumulatively assigned to the pixels as the pixel data.

The receiving section 330 may be configured to perform the reception beam-forming (i.e., summing) upon the sampling data which are cumulatively assigned to the respective pixels P_{a,b} of the ultrasound image UI to form the reception-focused data.

In another embodiment, the receiving section 330 may be configured to perform the analog-digital conversion upon the reception signals provided through the plurality of channels CHₖ from the ultrasound probe 310 to form the sampling data as shown in FIG. 8. The sampling data S_{i,j} may be stored in the storage unit 140. The receiving section 330 may be further configured to detect pixels corresponding to the sampling data based on the positions of the elements 311 and the orientation of the pixels of the ultrasound image UI with respect to the elements 311. That is, the receiving section 330 may select the pixels that the respective sampling data are used as the pixel data thereof, during the reception beam-forming based on the positions of the elements 311 and the orientation of the respective pixels of the ultrasound image UI with respect to the elements 311. The receiving section 330 may be configured to cumulatively assign the sampling data corresponding to the selected pixels as the pixel data. The receiving section 330 may be further configured to determine pixels existing in the same column among the selected pixels. The receiving section 330 may be further configured to set weights corresponding to the respective determined pixels. The receiving section 330 may be further configured to apply the weights to the sampling data of the respective pixels.

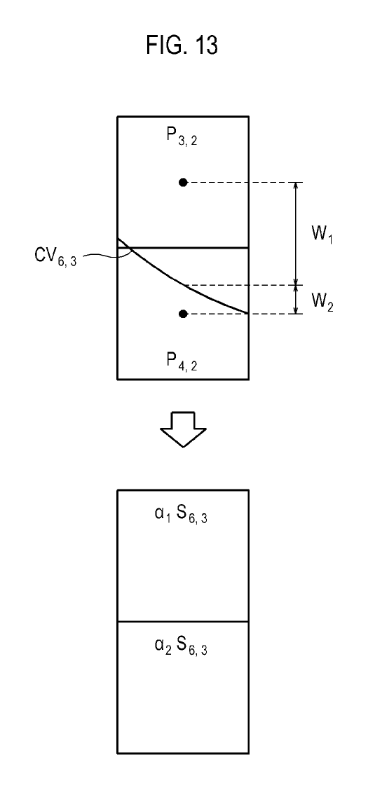

For example, the receiving section 330 may be configured to set the reception beam-forming curve CV_{6,3} for selecting pixels that the sampling data S_{6,3} are used as the pixel data thereof, during the reception beam-forming based on the positions of the elements 311 and the orientation of the respective pixels of the ultrasound image UI with respect to the elements 311, as shown in FIG. 9. The receiving section 330 may be further configured to detect the pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N} corresponding to the reception beam-forming curve CV_{6,3} from the pixels P_{a,b} of the ultrasound image UI. That is, the receiving section 330 may select the pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N} on which the reception beam-forming curve CV_{6,3} passes among the pixels P_{a,b} of the ultrasound image UI. The receiving section 330 may be further configured to assign the sampling data S_{6,3} to the selected pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N}, as shown in FIG. 10. The receiving section 330 may be further configured to determine pixels P_{3,2} and P_{4,2} which exist in the same column among the selected pixels P_{3,1}, P_{3,2}, P_{4,2}, P_{4,3}, P_{4,4}, P_{4,5}, P_{4,6}, P_{4,7}, P_{4,8}, P_{4,9}, . . . P_{3,N}. The receiving section 330 may be further configured to calculate a distance W₁ from a center of the determined pixel P_{3,2} to the reception beam-forming curve CV_{6,3} and a distance W₂ from a center of the determined pixel P_{4,2} to the reception beam-forming curve CV_{6,3}, as shown in FIG. 13. The receiving section 330 may be further configured to set a first weight α₁ corresponding to the pixel P_{3,2} based on the distance W₁ and a second weight α₂ corresponding to the pixel P_{4,2} based on the distance W₂. The first weight α₁ and the second weight α₂ may be set to be in proportion to or in inverse proportion to the calculated distances. The receiving section 330 may be further configured to apply the first weight α₁ to the sampling data S_{6,3} assigned to the pixel P_{3,2} and to apply the second weight α₂ to the sampling data S_{6,3} assigned to the pixel P_{4,2}. The receiving section 330 may be configured to perform the above process upon the remaining sampling data.

The receiving section 330 may be configured to perform the reception beam-forming upon the sampling data which are cumulatively assigned to the respective pixels P_{a,b} of the ultrasound image UI to form the reception-focused data.

In yet another embodiment, the receiving section 330 may be configured to perform the analog-digital conversion upon the reception signals provided through the plurality of channels CHₖ from the ultrasound probe 310 to form the sampling data S_{i,j}, as shown in FIG. 8. The sampling data S_{i,j} may be stored in the storage unit 140. The receiving section 330 may be further configured to set a sampling data set for selecting pixels that the sampling data S_{i,j} are used as the pixel data thereof, during the reception beam-forming.

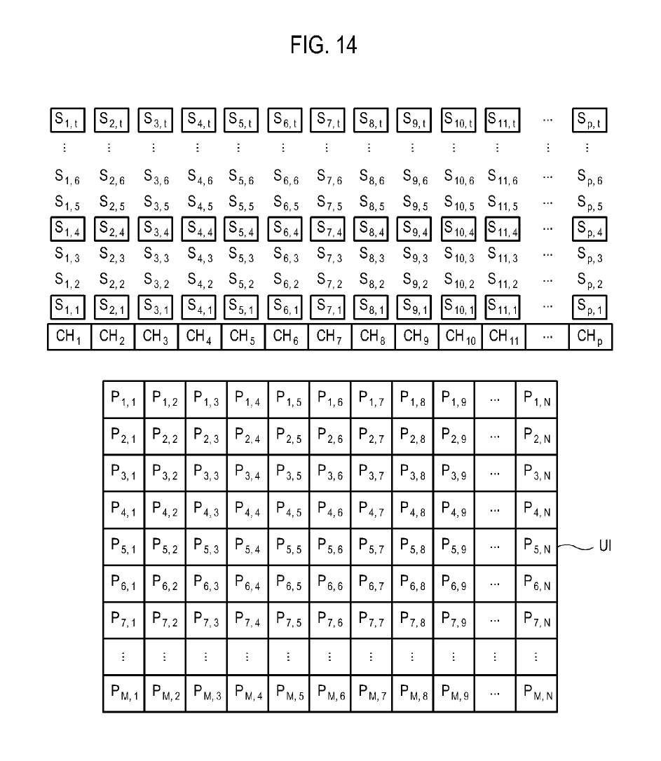

For example, the receiving section 330 may be configured to set the sampling data S_{1,1}, S_{1,4}, . . . S_{1,t}, S_{2,1}, S_{2,4}, . . . S_{2,t}, . . . S_{p,t} as the sampling data set (denoted by a box) for selecting the pixels that the sampling data S_{i,j} are used as the pixel data thereof, during the reception beam-forming, as shown in FIG. 14.

The receiving section 330 may be further configured to detect the pixels corresponding to the respective sampling data of the sampling data set based on the positions of the elements 311 and the orientation of the respective pixels of the ultrasound image UI with respect to the elements 311. That is, the receiving section 330 may select the pixels that the respective sampling data of the sampling data set are used as the pixel data thereof, during the reception beam-forming based on the positions of the elements 311 and the orientation of the respective pixels of the ultrasound image UI with respect to the elements 311. The receiving section 330 may be further configured to cumulatively assign the sampling data to the selected pixels in the same manner with the above embodiments. The receiving section 330 may be further configured to perform the reception beam-forming upon the sampling data which are cumulatively assigned to the respective pixels of the ultrasound image UI to form the reception-focused data.

In yet another embodiment, the receiving section 330 may be configured to perform a down-sampling upon the reception signals provided through the plurality of channels CHₖ from the ultrasound probe 310 to form down-sampling data. As described above, the receiving section 330 may be further configured to detect the pixels corresponding to the respective sampling data, based on the positions of the elements 311 and the orientation of the respective pixels of the ultrasound image UI with respect to the elements 311. That is, the receiving section 330 may select the pixels that the respective sampling data are used as the pixel data thereof, during the reception beam-forming based on the positions of the elements 311 and the orientation of the pixels of the ultrasound image UI with respect to the elements 311. The receiving section 330 may be further configured to cumulatively assign the respective sampling data to the selected pixels in the same manner of the above embodiments. The receiving section 330 may be further configured to perform the reception beam-forming upon the sampling data which are cumulatively assigned to the respective pixels of the ultrasound image UI to form the reception-focused data.

However, it should be noted herein that the reception beam-forming may not be limited thereto.

Referring back to FIG. 3, the ultrasound data acquiring unit 120 may further include an ultrasound data forming section 340. The ultrasound data forming section 340 may be configured to form the ultrasound data corresponding to the ultrasound image based on the reception-focused data provided from the receiving section 330. The ultrasound data forming section 340 may be further configured to perform a signal process (e.g., gain control, etc) upon the reception-focused data.

In one embodiment, the ultrasound data forming section 340 may be configured to form ultrasound data (hereinafter, referred to as “brightness mode ultrasound data”) corresponding to the brightness mode image based on the brightness mode reception-focused data provided from the receiving section 330. The brightness mode ultrasound data may include radio frequency data.

The ultrasound data forming section 340 may be further configured to form ultrasound data (hereinafter, referred to as “Doppler mode ultrasound data”) corresponding to the region of interest ROI based on the Doppler mode reception-focused data provided from the receiving section 330. The Doppler mode ultrasound data may include in-phase/quadrature data. However, it should be noted herein that the Doppler mode ultrasound data may not be limited thereto.

For example, the ultrasound data forming section 340 may form first Doppler mode ultrasound data based on the first Doppler mode reception-focused data provided from the receiving section 330. The ultrasound data forming section 340 may further form second Doppler mode ultrasound data based on the second Doppler mode reception-focused data provided from the receiving section 330.

Referring back to FIG. 1, the ultrasound system 100 may further include a processing unit 130 in communication with the user input unit 110 and the ultrasound data acquiring unit 120. The processing unit 130 may include a central processing unit, a microprocessor, a graphic processing unit and the like.

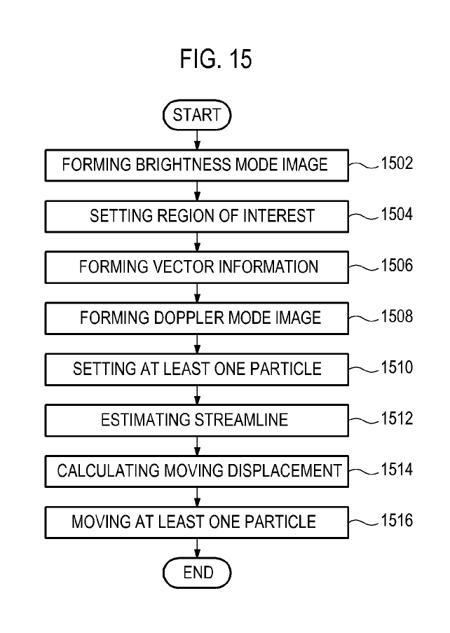

FIG. 15 is a flow chart showing a process of estimating motion of at least one particle. The processing unit 130 may be configured to form the brightness mode image BI based on the brightness mode ultrasound data provided from the ultrasound data acquiring unit 120, at step S1502 in FIG. 15. The brightness mode image BI may be displayed on a display unit 150. Thus, the user may set the region of interest ROI on the brightness mode image BI displayed on the display unit 150 by using the user input unit 110.

The processing unit 130 may be configured to set the region of interest ROI on the brightness mode image BI based on the input information (i.e., first input information) provided from the user input unit 110, at step S1504 in FIG. 15. Thus, the ultrasound data acquiring unit 120 may be configured to transmit the ultrasound signals to the living body and receive the ultrasound echo signals from the living body to acquire the Doppler mode ultrasound data, in consideration of the region of interest ROI.

The processing unit 130 may be configured to form vector information based on the Doppler mode ultrasound data provided from the ultrasound data acquiring unit 120, at step S1506 in FIG. 15. That is, the processing unit 130 may form the vector information corresponding to motion (i.e., velocity and direction) of the target object based on the Doppler mode ultrasound data.

Generally, when the transmission direction of the ultrasound signals is equal to the reception direction of the ultrasound echo signals and a Doppler angle is θ, the following relationship may be established:

$$X cos θ = \frac{{C}_{0} {f}_{d}}{2 {f}_{0}} \qquad ( 1 )$$

In Equation 1, X represents a reflector velocity (i.e., velocity of target object), C₀ represents a sound speed in the living body, f_{d} represents a Doppler shift frequency, and f₀ represents an ultrasound frequency.

The Doppler shift frequency f_{d} may be calculated by the difference between a frequency of the ultrasound signals (i.e., transmission beam) and a frequency of the ultrasound echo signals (i.e., reception beam). Also, the velocity component X cos θ projected to the transmission direction may be calculated by Equation 1.

When the transmission direction of the ultrasound signals (i.e., transmission beam) is different from the reception direction of the ultrasound echo signals (i.e., reception beam), the following relationship may be established:

$$X cos {θ}_{T} + X cos {θ}_{R} = \frac{{C}_{0} {f}_{d}}{{f}_{0}} \qquad ( 2 )$$

In Equation 2, θ_{T} represents an angle between the ultrasound signals (i.e., transmission beam) and the blood flow, and θ_{R} represents an angle between the ultrasound echo signals (i.e., reception beam) and the blood flow.

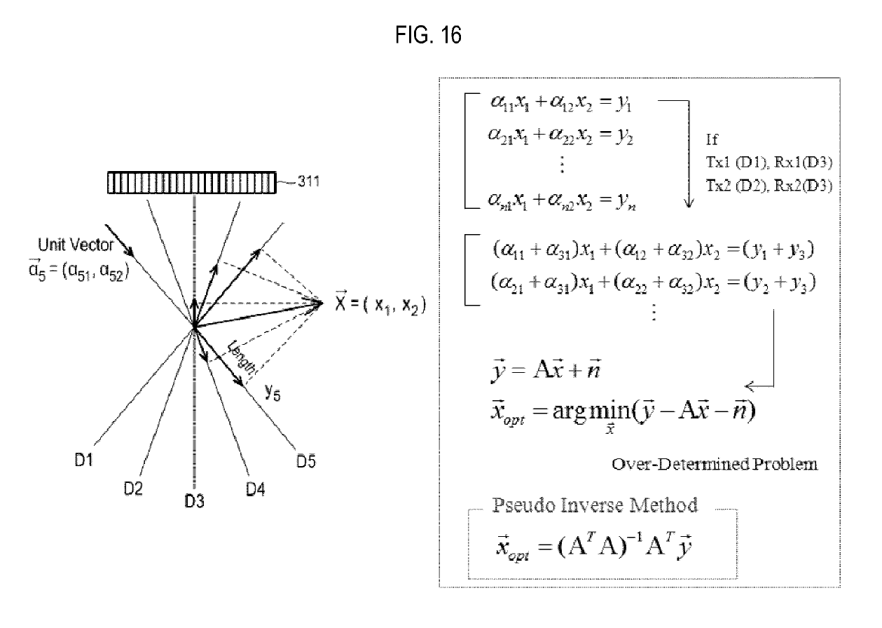

FIG. 16 is a schematic diagram showing an example of the transmission directions, the reception directions, the vector information and an over-determined problem. Referring to FIG. 16, when the ultrasound signals (i.e., transmission beam) are transmitted in a first direction D1 and the ultrasound echo signals (i.e., reception beam) are received in the first direction D1, the following relationship may be established:

\vec{α₁}\vec{X}=α₁₁x₁+α₁₂x₂=y₁=X cos θ (3)

In Equation 3, \vec{α₁}=(α₁₁,α₁₂) represents a unit vector of the first direction D1, \vec{X}=(x₁, x₂) represents variables, and y₁ is calculated by Equation 1.

When the ultrasound signals (i.e., transmission beam) are transmitted in a second direction D2 and the ultrasound echo signals (i.e., reception beam) are received in a third direction D3, the following relationship may be established:

(α₂₁+α₃₁)x₁+(α₂₂+α₃₂)x₂=(y₂+y₃)=X cos θ₂+X cos θ₃ (4)

Equations 3 and 4 may be set to assume two-dimensional environment. Further, Equations 3 and 4 may be expanded to three-dimensional environment. That is, when expanding Equations 3 and 4 to the three-dimensional environment, the following relationship may be established:

α₁₁x₁+α₁₂x₂+α₁₃x₃=y (5)

In the case of the two-dimensional environment (i.e., two-dimensional vector), at least two equations are required to calculate the variables x₁ and x₂. For example, when the ultrasound signals (i.e., transmission beam) are transmitted in the third direction D3 and the ultrasound echo signals (i.e., reception beam) are received in the second direction D2 and a fourth direction D4 as shown in FIG. 16, the following equations may be established:

(α₃₁+α₂₁)x₁+(α₃₂+α₂₂)x₂=(y₃+y₂)

(α₃₁+α₄₁)x₁+(α₃₂+α₄₂)x₂=(y₃+y₄) (6)

The vector \vec{X}=(x₁,x₂) may be calculated by the equations of Equation 6.

When the reception beam-forming is performed in at least two angles (i.e., at least two reception directions), at least two equations may be obtained and represented as the over-determined problem, as shown in FIG. 16. The over-determined problem may be solved by a pseudo inverse method, a weighted least square method and the like based on noise characteristics added to the Doppler shift frequency. The over-determined problem is well known in the art. Thus, it has not been described in detail so as not to unnecessarily obscure the present disclosure.

That is, M×N equations may be obtained by M transmission directions and the reception beam-forming of N reception directions at every transmission.

Referring back to FIG. 15, the processing unit 130 may be configured to form the Doppler mode image DMIₖ, wherein the k is a positive integer, based on the vector information, at step S 1508 in FIG. 15. The Doppler mode image DMIₖ may be displayed on the display unit 150. Thus, the user may set the at least one particle on the Doppler mode image DMIₖ displayed on the display unit 150 by using the user input unit 110.

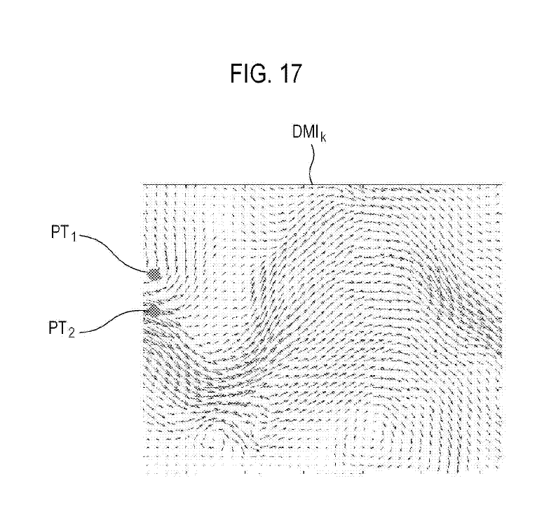

The processing unit 130 may be configured to set the at least one particle on the Doppler mode image DMIₖ based on the input information (i.e., second input information) provided from the user input unit 110, at step S1510 in FIG. 15. That is, the processing unit 130 may set the at least one particle on the Doppler mode image DMIₖ based on the second input information for setting the number, position, size, color and shape of the particle, an alignment position between the particles and the like. For example, the processing unit 130 may set the particles PT₁, PT₂ on the Doppler mode image DMIₖ based on the second input information provided from the user input unit 110, as shown in FIG. 17.

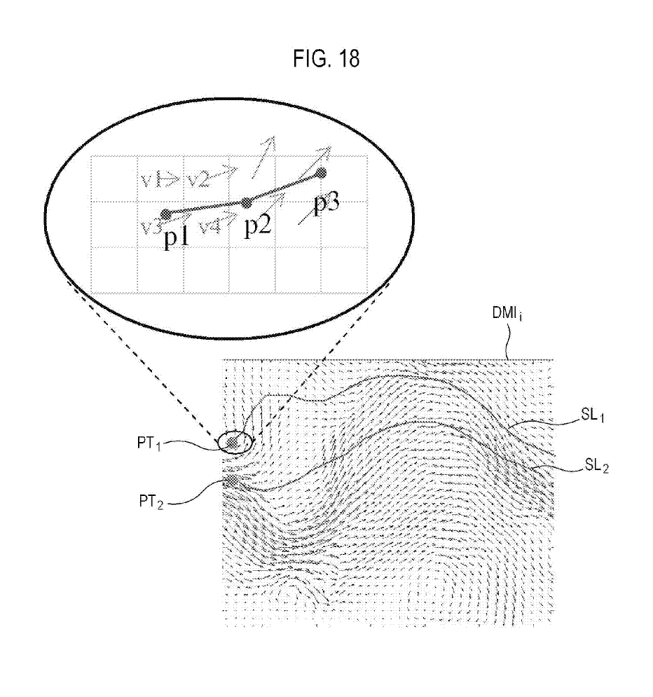

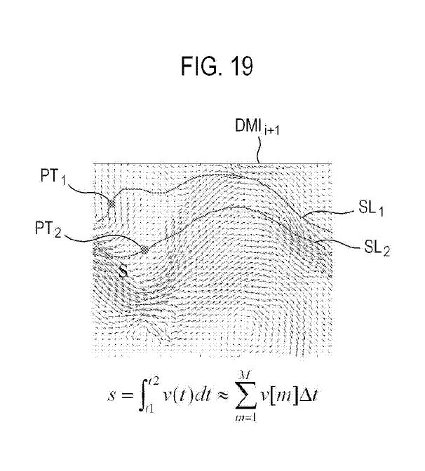

The processing unit 130 may be configured to estimate at least one streamline corresponding to motion of the at least one particle based on the vector information, at step S1512 in FIG. 15. For example, the processing unit 130 may interpolate vector information v1, v2, v3 and v4 adjacent to a first position p1 of the first particle PT₁ for an i^{th} Doppler mode image DMIᵢ, wherein the i is a positive integer, to estimate a stream direction at the first position p1, as shown in FIG. 18. The processing unit 130 may further estimate a second position p2 of the first particle PT₁ based on the stream direction and a predetermined moving displacement S with respect to the first position p1. The processing unit 130 may further interpolate vector information adjacent to the second position p2 of the first particle PT₁ to estimate a stream direction at the second position p2. The processing unit 130 may further estimate a third position p3 of the first particle PT1 based on the stream direction at the second position p2 and the predetermined moving displacement S with respect to the second position p2. As described above, the processing unit 130 may estimate a first streamline SL₁ corresponding to the first particle PT₁ and a second streamline SL₂ corresponding to the second particle PT₂ for the i^{th} Doppler mode image DMIᵢ. Thereafter, the processing unit 130 may estimate the first streamline SL₁ corresponding to the first particle PT₁ and the second streamline SL₂ corresponding to the second particle PT₂ for an (i+1)^{th} Doppler mode image DMIᵢ₊₁, as shown in FIG. 19.

Referring back to FIG. 15, the processing unit 130 may be configured to calculate a moving displacement of the at least one particle based on a time interval between adjacent Doppler mode images and the vector information (i.e., current velocity of at least one particle), at step S1514 in FIG. 15. For example, the processing unit 130 may calculate the moving displacement (s=v×dt) of the respective particles PT₁, PT₂ based on the time interval dt between the Doppler mode images DMIᵢ and DMIᵢ₊₁ and the velocity v at the current position of the respective particles PT₁, PT₂.

Optionally, the processing unit 130 may further calculate the moving displacement of the at least one particle in inverse proportion to a weight corresponding to the at least one particle, wherein the weight may be set by a user.

The processing unit 130 may be configured to move the at least one particle by a distance corresponding to the calculated moving displacement along the streamline set on the Doppler mode image DMIₖ, at step S1516 in FIG. 15. For example, the processing unit 130 may move the particle PT₁ along the first streamline SL₁ by the distance corresponding to the moving displacement of the particle PT₁ for the i^{th} Doppler mode image DMIᵢ₊₁, as shown in FIG. 19. The processing unit 130 may further move the particle PT₂ along the second streamline SL₂ by the distance corresponding to the moving displacement of the particle PT₂ for the Doppler mode image DMIᵢ₊₁, as shown in FIG. 19.

In another embodiment, the processing unit 130 may readjust the number and position of the particle at every Doppler mode image DMIₖ. For example, the processing unit 130 may set the N particles on the i^{th} Doppler mode image DMIᵢ, wherein the N is an integer two or greater. The processing unit 130 may further detect at least one particle, which does not move (i.e., moving displacement is 0), from the (i+1)^{th} Doppler mode image DMIᵢ₊₁. The processing unit 130 may further remove the detected particle. The processing unit may further set the number of a new particle corresponding to the removed particle on the (i+1)^{th} Doppler mode image DMIᵢ₊₁. Thus, the N particles may be set on the Doppler mode image DMIₖ.

In yet another embodiment, the processing unit 130 may relocate the position of the at least one particle on a line at every predetermined frame (i.e., Doppler mode image). In this way, a profile pattern corresponding to blood flow may be provided by relocating the position of the at least one particle on the line.

In still yet another embodiment, the processing unit 130 may set the at least one particle on the Doppler mode image DMIᵢ in synchronization with electrocardiogram signals. In this way, the motion of the at least one particle may be provided in synchronization with the electrocardiogram signals by setting the at least one particle in synchronization with the electrocardiogram signals.

In a further embodiment, the processing unit 130 may estimate the streamline corresponding to the at least one particle between the i^{th} Doppler mode image DMIᵢ and the (i+1)ₜₕ Doppler mode image DMIᵢ₊₁. That is, the processing unit 130 may further estimate the streamline by interpolating a vector field of the i^{th} Doppler mode image DMIᵢ and a vector field of the (i+1)^{th} Doppler mode image DMIᵢ₊₁.

In a still yet further embodiment, the processing unit 130 may estimate the motion of the at least one particle at every frame (i.e., Doppler mode image) or between the i^{th} Doppler mode image DMIᵢ and the (i+1)^{th} Doppler mode image DMIᵢ₊₁.

Referring back to FIG. 1, the ultrasound system 100 may further include the storage unit 140. The storage unit 140 may store the ultrasound data (i.e., brightness mode ultrasound data and Doppler mode ultrasound data) acquired by the ultrasound data acquiring unit 120. The storage unit 140 may further store the vector information formed by the processing unit 130.

The ultrasound system 100 may further include the display unit 150. The display unit 150 may be configured to display the brightness mode image BI formed by the processing unit 130. The display unit 150 may be further configured to display the Doppler mode image formed by the processing unit 130. The display unit 150 may be further configured to display the at least one particle.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
a processing unit configured to form vector information of a target object based on ultrasound data corresponding to the target object, form a plurality of Doppler mode images based on the vector information, set at least one particle on the Doppler mode images based on input information of a user, and estimate a motion of the at least one particle based on the vector information.

2. The ultrasound system of claim 1, further comprising:
an ultrasound data acquiring unit configured to transmit ultrasound signals to a living body including the target object in at least one transmission direction, and receive ultrasound echo signals from the living body in at least one reception direction to acquire the ultrasound data corresponding to the at least one reception direction.

3. The ultrasound system of claim 2, wherein the ultrasound data acquiring unit is configured to:
transmit the ultrasound signals to the living body in a first transmission direction; and
receive the ultrasound echo signals from the living body in a first reception direction and a second reception direction to acquire the ultrasound data corresponding to the respective first and second reception directions.

4. The ultrasound system of claim 2, wherein the ultrasound data acquiring unit is configured to:
transmit the ultrasound signals to the living body in a first transmission direction and a second transmission direction; and
receive the ultrasound echo signals from the living body in a first reception direction to acquire the ultrasound data corresponding to the first reception direction of the respective first and second transmission directions.

5. The ultrasound system of claim 2, wherein the ultrasound data acquiring unit is configured to:
transmit the ultrasound signals to the living body in a first transmission direction and a second transmission direction; and
receive the ultrasound echo signals from the living body in a first reception direction and a second reception direction to acquire the ultrasound data corresponding to the respective first and second reception directions.

6. The ultrasound system of claim 2, wherein the ultrasound data acquiring unit is configured to transmit the ultrasound signals in an interleaved transmission scheme.

7. The ultrasound system of claim 2, wherein the ultrasound signals include plane wave signals or focused signals.

8. The ultrasound system of claim 1, wherein the processing unit is configured to form the vector information corresponding to a velocity and a direction of the target object in consideration of at least one transmission direction and at least one reception direction corresponding to the at least one transmission direction based on the ultrasound data.

9. The ultrasound system of claim 1, wherein the processing unit is configured to:
estimate a streamline corresponding to a motion of the at least one particle based on the vector information;
calculate a moving displacement of the at least one particle based on the vector information and time interval between adjacent Doppler mode images; and
move the at least one particle along the streamline by a distance corresponding to the moving displacement.

10. The ultrasound system of claim 9, wherein the processing unit is configured to:
interpolate the vector information adjacent to an i^{th} position of the at least one particle to estimate a stream direction at the i^{th} position, wherein the i is a positive integer;
estimate an (i+1)^{th} position of the at least one particle based on the stream direction and a predetermined moving displacement with respect to the i^{th} position to estimate the streamline.

11. The ultrasound system of claim 10, wherein the processing unit is configured to calculate the moving displacement based on the time interval between an i^{th} Doppler mode image and an (i+1)^{th} Doppler mode image and a velocity at a current position of the at least one particle.

12. The ultrasound system of claim 10, wherein the processing unit is further configured to calculate the moving displacement in an inverse proportion to a weight corresponding to the at least one particle.

13. The ultrasound system of claim 11, wherein the processing unit is further configured to readjust the number and position of the particle at every Doppler mode image.

14. The ultrasound system of claim 13, wherein the processing unit is further configured to:
detect at least one non-moving particle from the i^{th} Doppler mode image;
remove the detected particle from the (i+1)^{th} Doppler mode image;
set the number of new particle corresponding to the removed particle on the (i+1)^{th} Doppler mode image.

15. The ultrasound system of claim 1, wherein the processing unit is further configured to relocate the position of the at least one particle on a line at every predetermined Doppler mode image.

16. The ultrasound system of claim 1, wherein the processing unit is further configured to set the at least one particle on the Doppler mode images in synchronization with electrocardiogram signals.

17. The ultrasound system of claim 11, wherein the processing unit is further configured to estimate the streamline corresponding to the at least one particle between the i^{th} Doppler mode image and the (i+1)^{th} Doppler mode image.

18. The ultrasound system of claim 11, wherein the processing unit is further configured to estimate motion of the at least one particle at every Doppler mode image or between an i^{th} Doppler mode image and an (i+1)^{th} Doppler mode image.

19. A method of estimating motion of at least one particle, comprising:
a) forming vector information of a target object based on ultrasound data corresponding to the target object;
b) forming a plurality of Doppler mode images based on the vector information;
c) setting at least one particle on the Doppler mode images based on input information of a user; and
d) estimating a motion of the at least one particle based on the vector information.

20. The method of claim 19, further comprising:
transmitting ultrasound signals to a living body including the target object in at least one transmission direction and receiving ultrasound echo signals from the living body in at least one reception direction to acquire the ultrasound data corresponding to the at least one reception direction, prior to performing the step a).

21. The method of claim 20, wherein the step of acquiring the ultrasound data comprises:
transmitting the ultrasound signals to the living body in a first transmission direction; and
receiving the ultrasound echo signals from the living body in a first reception direction and a second reception direction to acquire the ultrasound data corresponding to the respective first and second reception directions.

22. The method of claim 20, wherein the step of acquiring the ultrasound data comprises:
transmitting the ultrasound signals to the living body in a first transmission direction and a second transmission direction; and
receiving the ultrasound echo signals from the living body in a first reception direction to acquire the ultrasound data corresponding to the first reception direction of the respective first and second transmission directions.

23. The method of claim 20, wherein the step of acquiring the ultrasound data comprises:
transmitting the ultrasound signals to the living body in a first transmission direction and a second transmission direction; and
receiving the ultrasound echo signals from the living body in a first reception direction and a second reception direction to acquire the ultrasound data corresponding to the respective first and second reception directions.

24. The method of claim 20, wherein the ultrasound signals are transmitted in an interleaved transmission scheme.

25. The method of claim 20, wherein the ultrasound signals include plane wave signals or focused signals.

26. The method of claim 19, wherein the step a) comprises:
forming the vector information corresponding to a velocity and a direction of the target object in consideration of at least one transmission direction and at least one reception direction corresponding to the at least one transmission direction based on the ultrasound data.

27. The method of claim 19, wherein the step d) comprises:
d1) estimating a streamline corresponding to a motion of the at least one particle based on the vector information;
d2) calculating a moving displacement of the at least one particle based on the vector information and time interval between adjacent Doppler mode images; and
d3) moving the at least one particle along the streamline by a distance corresponding to the moving displacement.

28. The method of claim 27, wherein the step d1) comprises:
interpolating the vector information adjacent to an i^{th} position of the at least one particle to estimate a stream direction at the i^{th} position, wherein the i is a positive integer;
estimating an (i+1)^{th} position of the at least one particle based on the stream direction and a predetermined moving displacement with respect to the i^{th} position to estimate the streamline.

29. The method of claim 27, wherein the step d2) comprises:
calculating the moving displacement based on the time interval between an i^{th} Doppler mode image and an (i+1)^{th} Doppler mode image and a velocity at a current position of the at least one particle.

30. The method of claim 27, wherein the step d2) further comprises:
calculating the moving displacement in an inverse proportion to a weight corresponding to the at least one particle, wherein the weight is set by a user.

31. The method of claim 26, further comprising:
e) readjusting the number and position of the particle at every Doppler mode image.

32. The method of claim 31, wherein the step e) further comprises:
detecting at least one non-moving particle from the i^{th} Doppler mode image;
removing the detected particle from the (i+1)^{th} Doppler mode image;
setting the number of new particle corresponding to the removed particle on the (i+1)^{th} Doppler mode image.

33. The method of claim 19, further comprising:
e) relocating the position of the at least one particle on a line at every predetermined Doppler mode image.

34. The method of claim 19, wherein the step c) further comprises:
setting the at least one particle on the Doppler mode images in synchronization with electrocardiogram signals.

35. The method of claim 29, wherein the step d) further comprises:
estimating the streamline corresponding to the at least one particle between the i^{th} Doppler mode image and the (i+1)^{th} Doppler mode image.

36. The method of claim 19, wherein the step d) further comprises:
estimating motion of the at least one particle at every Doppler mode image or between an i^{th} Doppler mode image and an (i+1)^{th} Doppler mode image.
